(19) United States
(12) Patent Application Publication
(10) Pub. No.: US 2018/0318477 A1
(43) Pub. Date: Nov. 8, 2018

(54) WOUND THERAPY

(71) Applicant: Ehren Cronje Eksteen, Montana, Pretoria (ZA)

(72) Inventor: Ehren Cronje Eksteen, Montana, Pretoria (ZA)

(21) Appl. No.: 15/776,127

(22) PCT Filed: May 15, 2017
(86) PCT No.: PCT/IB2017/052847

§ 371 Date: May 15, 2018

(30) Foreign Application Priority Data
May 13, 2016 (ZA) 2016/03245

(51) Int. Cl. A61M 1/00; A61M 35/00; A61F 13/02; A61M 39/10

(52) U.S. Cl. CPC A61M 1/0086; A61M 1/0084; A61M 35/00; A61M 1/0088; A61M 2039/1027; A61M 39/10; A61F 13/0216; A61M 2039/1072; A61F 13/0206

(57) ABSTRACT

The invention relates to a wound bed access device for providing access to a wound bed from outside a closed wound. The wound bed access device includes a self-sealing valve connector defining a flow path which is ordinarily closed and is opened when a complementary opening connector is connected to the self-sealing valve connector. The wound bed access device also includes a wound dressing interface, shaped and dimensioned to be closed within a wound dressing covering a wound. The self-sealing valve connector is connected to the wound dressing interface, with the flow path passing from the self-sealing valve connector through the wound dressing interface. In use, the self-sealing valve connector is positioned outside the wound dressing thereby providing access to the wound bed from outside the closed wound.

This invention relates to wound therapy. In particular the invention relates to a wound bed access device, a negative pressure treatment device and a wound care system.

### BACKGROUND INFORMATION

There are various techniques that can be applied in treating wounds and one such technique is Negative Pressure Wound Therapy (NPWT). This technique is used to treat chronic wounds or wounds that are expected to present difficulty when healing by creating a moist environment which promotes the wound healing process.

The technique involves applying negative pressure to the wound by applying a vacuum through a sealed dressing, either continuously or intermittently. The vacuum serves to draw fluid, which may contain infectious materials, from the wound and increases blood flow to the area. The negative pressure also helps to draw the edges of the wound together. The sealed dressing is connected to a drainage tube, which removes the fluid absorbed by the dressing and transfers it to a canister.

NPWT is not without adverse effects, such as pain, bleeding, infection, other complications. Proper monitoring of the patient and wound, as well as regular changes of the dressing, can help to decrease the risk of any adverse effects.

The inventor is aware of the technique and the possible adverse effects associated with it and aims to provide an invention which will improve the current use of NPWT.

### SUMMARY OF THE INVENTION

Broadly according to a first aspect of the invention there is provided a wound bed access device for providing access to a wound bed from outside a closed wound, which includes

a self-sealing valve connector defining a flow path which is ordinarily closed and is opened when a complementary opening connector is connected to the self-sealing valve connector; and

a wound dressing interface, shaped and dimensioned to be enclosed within a wound dressing covering a wound, the self-sealing valve connector being connected to the wound dressing interface, with the flow path passing from the self-sealing valve connector through the wound dressing interface, in use the self-sealing valve connector is positioned outside the wound dressing thereby providing access to the wound bed from outside the closed wound.

In one embodiment, the self-sealing valve connector may be distal from the wound dressing interface, the self-sealing valve connector and wound dressing interface having a distal access tube extending between them.

The distal access tube may be detachable from any one or both of the self-sealing valve connector and wound dressing interface. Alternatively, the wound bed access device may be of unitary construction.

The wound dressing interface may be in the form of a flexible pad, the flexible pad providing a planar surface for a wound cover to adhere over. The flexible pad may be planar and extend perpendicularly to the flow path through the self-sealing valve connector. The flexible pad may have an underside which in use faces the wound bed and a top side having a planar surface over which the wound cover is placed. The self-sealing valve connector may be connected to the top side of the wound dressing interface, such that in use the wound dressing interface can be sealed within the wound dressing covering the wound, such that the self-sealing valve connector is outside the sealed wound.

The self-sealing valve connector may ordinarily be in a closed position. The self-sealing valve connector may be connectable to the complementary opening connector, which upon connection with the self-sealing valve connector, opens the valve connector to allow flow through the valve connector. When the complementary opening connector is disconnected from the self-sealing valve connector, the valve connector returns to a closed position, preventing flow there through.

The self-sealing valve connector may be in the form of a quick-disconnect coupler.

The self-sealing valve connector may be in the form of a luer type connector.

In one embodiment the self-sealing valve connector may be in the form of a female luer access connector. In such an embodiment the opening connector may be in the form of male luer taper. In another embodiment the self-sealing valve connector may be in the form of a male luer connector and the opening connector may be in the form of a female luer connector.

The self-sealing valve connector may include a movable sealing member that controls the flow of fluid and/or gas through the self-sealing valve connector.

The movable sealing member may be longitudinally movable within the valve connector. The self-sealing valve connector may include a biasing member such as a spring for keeping the sealing member in an ordinarily closed position. When the opening connector is connected to the self-sealing valve connector the movable sealing member is pushed down providing a flow path through the self-sealing valve connector.

The movable sealing member may be in the form of any one of a ball, a sphere, a diaphragm, a flap or the like.

In one example the movable sealing member may be in the form of a hinged flap.

The hinged flap may be movable within the valve connector. The hinged flap is ordinarily in a closed position, in which the flap closes the flow path, and in which the flap extends transversely to the flow path. When the opening connector is connected to the self-sealing valve connector the flap is pushed down providing a flow path through the self-sealing valve connector.

In another embodiment the self-sealing valve connector may include a septum having a longitudinal slit. The opening connector may push through the slit when connected to the self-sealing valve connector, providing a flow path. When the opening connector is disconnected from the self-sealing valve connector, the slit in the septum closes and the flow path is closed off.

The wound bed access device may further include a wound bed extension tube, extending from the wound dressing interface on an opposed side to the self-sealing valve connector, being in flow communication with the flow path, in use to connect the self-sealing valve connector with the wound bed.

The wound bed extension tube may include a plurality of apertures, longitudinal grooves or the like, spaced along the length of the extension tube, through which fluid and/or gas may move. In use, when fluid and/or gas is introduced through the self-sealing valve connector, the fluid and/or gas is distributed to the wound bed through the plurality of apertures. Alternatively, when fluid and/or wound debris is extracted from the self-sealing valve connector, the fluid and/or wound debris is drawn in through the plurality of apertures.

In one embodiment, the wound bed extension tube may be connectable to the distal access tube at the wound dressing interface, such that in use the flow path extends through the self-sealing valve connector, distal access tube, wound dressing interface and wound bed extension tube into the wound. Alternatively, the distal access tube and wound bed extension tube may be of a single tube passing through the wound dressing interface, a portion of the tube extending operatively above the wound dressing interface defining the distal access tube and the portion operatively below the wound dressing interface defining the wound bed extension tube.

In accordance with another aspect of the invention, there is provided a negative pressure treatment device, which includes

a self-sealing valve connector defining a flow path which is ordinarily closed and is opened when a complementary opening connector is connected to the self-sealing valve connector;

a wound dressing interface, shaped and dimensioned to be enclosed within a wound dressing covering a wound, the self-sealing valve connector being connected to the wound dressing interface, with the flow path passing from the self-sealing valve connector through the wound dressing interface, in use the self-sealing valve connector is positioned outside the wound dressing thereby providing access to a wound bed from outside the closed wound; and

a reduced pressure port being integrated with the wound dressing interface.

The reduced pressure port may be connectable to a reduced pressure source, such as a vacuum pump.

The negative pressure treatment device may be operable to provide negative pressure and fluid and/or gas supply to the wound.

According to a further aspect of the invention, there is provided a wound care system, which includes

a wound bed access device as described, positioned proximate a wound; and

an airtight wound dressing, in use adhering around the self-sealing valve connector over the wound dressing interface, to seal the wound bed access device onto the wound.

The airtight wound dressing may be in the form of an adhesive film, surgical drape or the like. The airtight wound dressing may be adhered to intact skin peripheral to the wound and to the wound dressing interface of the wound bed access device, to provide an airtight seal around the wound.

In one embodiment, the wound care system may further include a reduced pressure port, sealed through the airtight seal around the wound. The reduced pressure port may be connected to the airtight wound dressing to provide flow communication to the sealed space created between the wound and the wound dressing. The reduced pressure port may be connectable to a reduced pressure source.

The airtight seal may be a planar sheet with two apertures for receiving the self-sealing valve connector and reduced pressure port there through, and sealing them over the wound.

In another embodiment, the wound bed access device is integrated into a negative pressure treatment device as described, which includes the wound bed access device, a wound bed extension tube and a reduced pressure port which is integrated with the wound dressing interface, the negative pressure treatment device being operable to be sealed over the wound. The wound dressing interface may be in the form of a flexible pad.

The wound care system may further include an absorbent insert. The absorbent insert may be in the form of any of: foam, gauze or the like. In a preferred embodiment the absorbent insert may be in the form of a foam pad. The foam pad may be of open cell polyurethane, polyether foam or the like. The adsorbent insert may be shaped to match the shape of the wound cavity. In use, the wound dressing interface is placed on the absorbent insert and the airtight wound dressing is sealed there over.

The wound care system may further include one or more supply apparatuses, for supplying gas and/or liquid to the wound bed. The supply apparatus may be connectable to the self-sealing valve connector, via an opening connector. The supply apparatus may be in the form of a fluid drip line, a gas line, a syringe or the like. The supply apparatus may include a pump, for forcing the gas and/or liquid through the self-sealing valve connector. The fluid drip line may be operable to administer medication or the like to the wound site. Examples of the medication may be in the form of any one or more of: antibiotics, pain relief medication, anti-inflammatories, or the like. The gas line may be operable to administer gas to the wound site. Examples of the gas may be in the form of a medicinal gas, O2, O3 or the like. The supply apparatus may intermittently be connected or disconnected from the self-sealing valve connector.

The wound care system may also include an extraction apparatus connectable to the self-sealing valve connector, for extracting wound fluid and/or debris from the wound bed. The extraction apparatus may be in the form of a syringe. The extraction apparatus may intermittently be connected or disconnected from the self-sealing valve connector.

The wound care system may include a receptacle connectable to the extraction apparatus, alternatively to the reduced pressure port for collecting wound fluid and/or debris which has been extracted from the wound bed. The receptacle may be disconnected from the extraction apparatus to dispose of the wound fluid and/or wound debris before being replaced. Alternatively, the receptacle may be in the form of a disposable receptacle.

The wound care system may include a reduced pressure source. The reduced pressure source may be in the form of a vacuum pump connected to the reduced pressure port for providing negative pressure at the wound site.

The vacuum pump may fluctuate the pressure, such that the absorbent insert switches between soaking and emptying. In another example the vacuum pump may draw a vacuum continuously.

The invention is now described, by way of non-limiting example, with reference to the accompanying drawings:

### DRAWING(S)

In the drawings:

FIG. 1 shows a three dimensional view of a wound bed access device in accordance with a first aspect of the invention according to one embodiment;

FIG. 2 shows shows a sectional view of a wound bed access device in accordance with a first aspect of the invention according to another embodiment;

FIG. 3 shows a sectional view of a wound care system in use;

FIG. 4 shows a sectional view of a negative pressure treatment device in accordance with a further aspect of the invention;

FIG. 5 shows another example of a wound care system.

In the drawings, like reference numerals denote like parts of the invention unless otherwise indicated.

### EMBODIMENT OF THE INVENTION

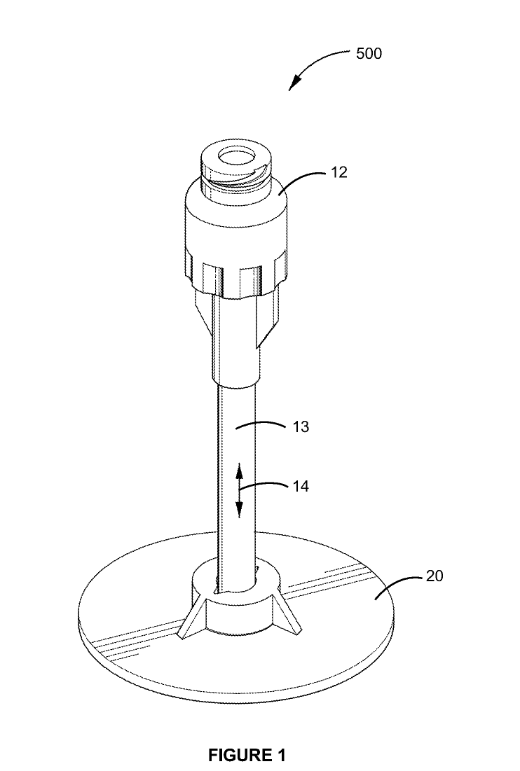

In FIG. 1 reference numeral (500) refers to a wound bed access device providing access to a wound bed from outside a sealed wound. The wound bed access device (500) includes a self-sealing valve connector (12) defining a flow path (14) which is ordinarily closed and is opened when a complementary opening connector is connected to the self-sealing valve connector (12). The wound bed access device (500) also includes a wound dressing interface (20) which is distal to the self-sealing valve connector, the self-sealing valve connector (12) and the wound dressing interface (20) having a distal access tube (13) extending between them, with the flow path (14) passing from the self-sealing valve connector (12) through the wound dressing interface (20).

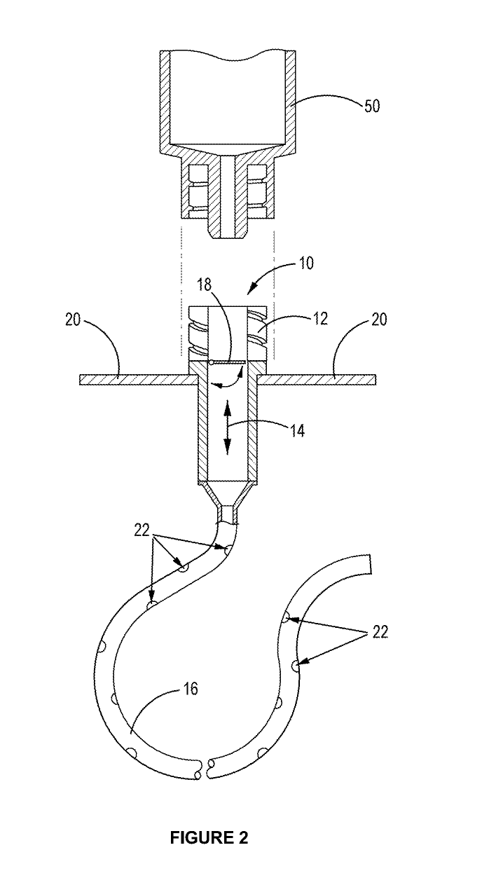

In FIG. 2 reference numeral (10) refers to another embodiment of the wound bed access device (10). The self-sealing valve connector (12) defining a flow path (14) that is ordinarily closed and is opened when a complementary opening connector (50) is connected to the self-sealing valve connector (12). In this embodiment, the self-sealing valve connector (12) has a wound bed extension tube (16) extending from it and is in flow communication with the flow path (14), in use to connect the self-sealing valve connector (12) with the wound bed.

As shown in FIG. 2, the self-sealing valve connector (12) is ordinarily in a closed position. The self-sealing valve connector (12) is connectable to a complementary opening connector (50), which upon connection with the self-sealing valve connector (12), opens the self-sealing valve connector (12) to allow flow through the valve. When the complementary opening connector (50) is disconnected from the self-sealing valve connector (12), the valve connector (12) returns to a closed position, preventing flow there through.

The self-sealing valve connector (12) includes a movable sealing member (18) that controls the flow of fluid and/or gas through the self-sealing valve connector (12). In this example the movable sealing member (18) is in the form of a hinged flap (18).

The hinged flap (18) is movable within the self-sealing valve connector (12). The hinged flap (18) is ordinarily in a closed position, in which the flap (18) closes the flow path (14), and in which the flap extends transversely to the flow path (14). When the opening connector (50) is connected to the self-sealing valve connector (12) the flap (18) is pushed down providing a flow path (14) through the self-sealing valve connector (12).

In this example, the wound dressing interface (20) is in the form of a flexible pad into which the self-sealing valve connector (12) is integrated. The flexible pad (20) provides a planar surface for a wound cover to adhere over, the flexible pad (20) being planar and extending perpendicularly to the flow path through the self-sealing valve connector (12). In this example the flexible pad (20) is of silicone, is about 3 mm thick and approximately 6 cm wide.

The wound bed extension tube (16) includes a plurality of apertures (22) spaced along the length of the extension tube (16), through which fluid and/or gas can move. In use, when fluid and/or gas is introduced through the self-sealing valve connector (12), the fluid and/or gas is distributed to the wound bed through the plurality of apertures (22). Alternatively, when fluid and/or wound debris is extracted from the self-sealing valve connector (12), the fluid and/or wound debris is drawn in through the plurality of apertures (22).

In this example the wound bed extension tube (16) is of Polyvinyl

Chloride (PVC) tubing. The wound bed extension tube (16) is about 20 cm long and includes ten apertures (22) spaced 1 cm apart.

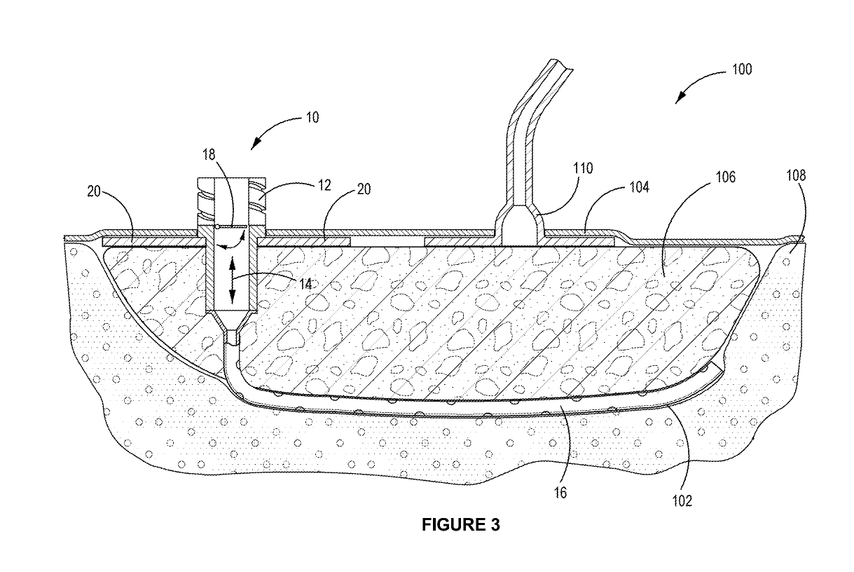

As shown in FIG. 3, according to a further aspect of the invention there is provided a wound care system (100). The wound care system (100) includes a wound bed access device (10) as described, positioned proximate a wound, with a wound bed extension tube (16) positioned in the wound bed (102) of the wound and an airtight wound dressing (104) adhering around the self-sealing valve connector (12) over the wound dressing interface (20), to seal the wound bed access device (10) onto the wound.

The wound care system (100) further includes an absorbent insert (106). In this example the absorbent insert (106) is in the form of a foam pad. The foam pad (106) is of open cell polyurethane. The foam pad (106) is shaped to match the shape of the wound cavity.

In this example the airtight wound dressing (104) is in the form of an adhesive film. In use, the airtight wound dressing (104) is adhered to intact skin (108) peripheral the wound site and to the flexible pad (20) of the wound bed access device (10), to provide an airtight seal around the wound.

In this example the wound care system (100) further includes a reduced pressure port (110), sealed through the airtight seal around the wound. The reduced pressure port (110) is connected to the airtight wound dressing (104) to provide flow communication to the sealed space created between the wound and the wound dressing (104).

The reduced pressure port (110) is connectable to a reduced pressure source, such as a vacuum pump.

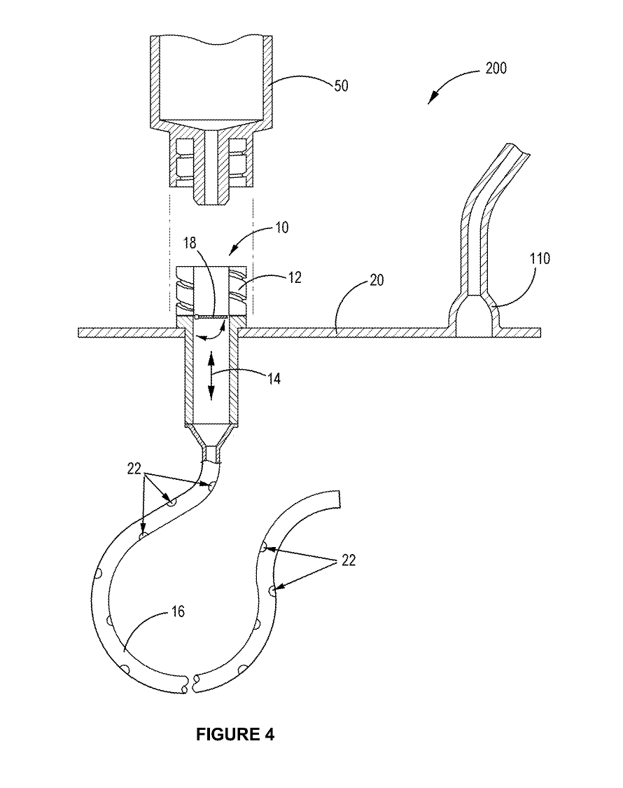

FIG. 4 shows a negative pressure treatment device (200), which includes a wound bed access device (10) as described, a wound bed extension tube (16) and a reduced pressure port (110) which is integrated into the flexible pad (20), the negative pressure treatment device (200) being operable to be sealed over a wound.

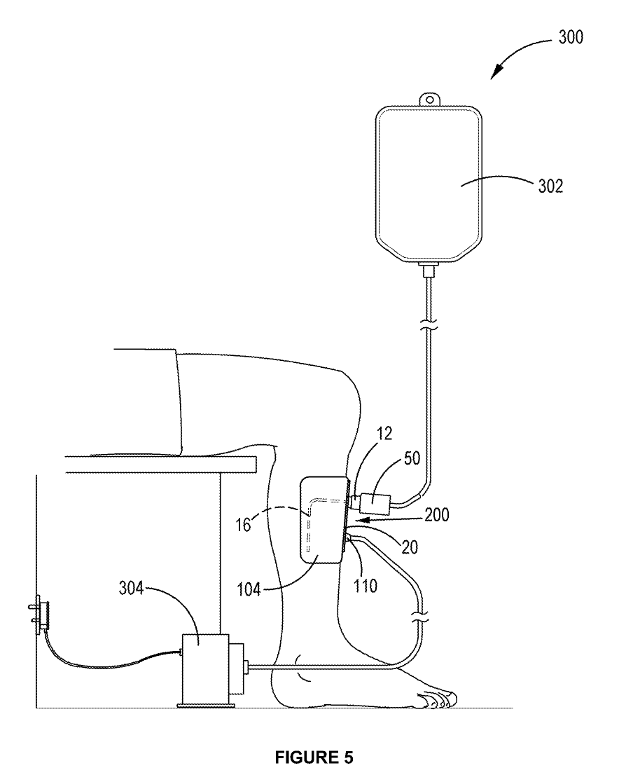

FIG. 5 shows a wound care system (300), which includes a wound bed access device (10) as described positioned on a wound with a wound bed extension tube (16) positioned in the wound bed of the wound and an airtight wound dressing (104) adhering around the self-sealing valve connector (12), to seal the wound bed access device (10) onto the wound.

In this example the wound bed access device (10) is integrated into the negative pressure treatment device (200) as shown in FIG. 4, which includes the wound bed access device (10), a wound bed extension tube (16) and a reduced pressure port (110) which is integrated into the flexible pad (20), the negative pressure treatment device (200) is sealed over the wound.

The wound care system (300) includes an absorbent insert (not shown). The absorbent insert is in the form of a foam pad of open cell polyurethane, which is shaped to match the shape of the wound cavity.

The airtight wound dressing (104) is in the form of an adhesive film which is adhered to intact skin peripheral the wound site and to the flexible pad (20) of the negative pressure treatment device (200).

The wound care system (300) further includes supply apparatuses, for supplying gas and/or liquid to the wound bed. The supply apparatus is connectable to the self-sealing valve connector (12), via an opening connector (50).

In this example the supply apparatus is in the form of a fluid drip line (302), but the fluid drip line (302) may be disconnected from the self-sealing valve connector (12) and switched with a gas line, syringe or the like.

The fluid drip line (302) is operable to administer medication or the like to the wound site. Examples of the medication may be in the form of any one or more of: antibiotics, pain relief medication, anti-inflammatories or the like.

The wound care system (300) also includes an extraction apparatus (not shown) connectable to the self-sealing valve connector (12), for extracting wound fluid and/or debris from the wound bed. In use, the extraction device is operable to extract samples from the wound, such that the microbial load or the like can be tested at time intervals without opening the wound.

The wound care system (300) also includes a reduced pressure source (304). In this example the reduced pressure source (304) is in the form of a vacuum pump having a storage canister connected to the reduced pressure port (110) for providing negative pressure at the wound site.

In this example the vacuum pump (304) fluctuates the pressure, such that the absorbent insert (not shown) switches between soaking and emptying.

The inventor believes that the invention provides a new negative pressure treatment device being operable to provide negative pressure, fluid and/or gas supply to the wound site and access to the wound for extracting samples.

1-43. (canceled)

44. A wound bed access device for providing access to a wound bed from outside a closed wound, the wound bed access device comprising:
a self-sealing valve connector defining a flow path which is ordinarily closed and is opened when a complementary opening connector is connected to the self-sealing valve connector; and
a wound dressing interface, shaped and dimensioned to be enclosed within a wound dressing covering a wound, the self-sealing valve connector being connected to the wound dressing interface, with the flow path passing from the self-sealing valve connector through the wound dressing interface, in use the self-sealing valve connector is positioned outside the wound dressing thereby providing access to the wound bed from outside the closed wound.

45. The wound bed access device as claimed in claim 44, which includes a distal access tube which extends between the self-sealing valve connector and wound dressing interface.

46. The wound bed access device as claimed in claim 44, in which the wound dressing interface is in the form of a flexible pad providing a planar surface for a wound cover to adhere over, the flexible pad being planar and extending perpendicularly to the flow path through the self-sealing valve connector.

47. The wound bed access device as claimed in claim 44, in which the complementary opening connector opens the self-sealing valve connector upon connection with the self-sealing valve connector, to allow flow through the valve connector and in which the self-sealing valve connector returns to a closed position when the complementary opening connector is disconnected from the self-sealing valve connector, preventing flow there through.

48. The wound bed access device as claimed in claim 44, in which the self-sealing valve connector is in the form of a quick-disconnect coupler.

49. The wound bed access device as claimed in claim 44, in which the self-sealing valve connector is in the form of a luer type connector.

50. The wound bed access device as claimed in claim 49, in which the luer type connector is in the form of a female luer access connector and the opening connector is in the form of a male luer taper.

51. The wound bed access device as claimed in claim 49, in which the luer type connector is in the form of a male luer connector and the opening connector is in the form of a female luer connector.

52. The wound bed access device as claimed in claim 44, in which the self-sealing valve connector includes a movable sealing member that controls the flow of fluid and/or gas through the self-sealing valve connector, the movable sealing member being movable within the self-sealing valve connector, and the self-sealing valve connector further includes a biasing member for keeping the movable sealing member in an ordinarily closed position, such that when the opening connector is connected to the self-sealing valve connector the movable sealing member is pushed down to provide a flow path through the self-sealing valve connector.

53. The wound bed access device as claimed in claim 44, in which the self-sealing valve connector includes a septum having a longitudinal slit and in which in use the opening connector pushes through the slit when connected to the self-sealing valve connector, providing a flow path and when the opening connector is disconnected from the self-sealing valve connector, the slit in the septum closes and the flow path is closed off.

54. The wound bed access device as claimed in claim 44, in which the device includes a wound bed extension tube, extending from the self-sealing valve connector and in flow communication with the flow path, in use to connect the self-sealing valve connector with a wound bed.

55. The wound bed access device as claimed in claim 54, in which the wound bed extension tube includes a plurality of apertures and longitudinal grooves spaced along the length of the extension tube, through which fluid and/or gas move, in use fluid and/or gas is introduced through the self-sealing valve connector, the fluid and/or gas being distributed to the wound bed through the plurality of apertures or fluid and/or wound debris is extracted from the self-sealing valve connector, the fluid and/or wound debris being drawn in through the plurality of apertures.

56. The wound bed access device as claimed in claim 45, in which the device includes a wound bed extension tube, extending from the self-sealing valve connector and in flow communication with the flow path, the wound bed extension tube being connectable to the distal access tube at the wound dressing interface, such that in use the flow path extends through the self-sealing valve connector, distal access tube, wound dressing interface and wound bed extension tube into the wound.

57. The wound bed access device as claimed in claim 45, in which the device includes a wound bed extension tube, extending from the self-sealing valve connector and in flow communication with the flow path, the wound bed extension and the distal access tube being of a single tube passing through the wound dressing interface, a portion of the tube extending operatively above the wound dressing interface defining the distal access tube and the portion operatively below the wound dressing interface defining the wound bed extension tube.

58. A negative pressure treatment device comprising:
a self-sealing valve connector defining a flow path which is ordinarily closed and is opened when a complementary opening connector is connected to the self-sealing valve connector;
a wound dressing interface, shaped and dimensioned to be enclosed within a wound dressing covering a wound, the self-sealing valve connector being connected to the wound dressing interface, with the flow path passing from the self-sealing valve connector through the wound dressing interface, in use the self-sealing valve connector is positioned outside the wound dressing thereby providing access to the wound bed from outside the closed wound; and
a reduced pressure port being integrated with the wound dressing interface, the reduced pressure port being connectable to a reduced pressure source;
wherein the negative pressure treatment device is operable to provide negative pressure and fluid and/or gas supply to the wound.

59. A wound care system comprising:
the wound bed access device of claim 44, positioned proximate a wound; and
an airtight wound dressing, in use adhering around the self-sealing valve connector over the wound dressing interface, to seal the wound bed access device onto the wound.

60. The wound care system as claimed in claim 59, in which the system includes a separate reduced pressure port sealable through the airtight wound dressing around the wound, the reduced pressure port being connectable through the airtight wound dressing to provide flow communication to the sealed space created between the wound and the wound dressing, and the reduced pressure port being connectable to a reduced pressure source.

61. The wound care system as claimed in claim 59, in which the wound bed access device includes a reduced pressure port integrated with the wound dressing interface, such that the wound dressing interface defines two flow paths there through, a first flow path associated with the self-sealing valve connector and a second flow path associated with the reduced pressured port.

62. The wound care system as claimed in claim 59, in which the wound care system includes an absorbent insert shaped to match the shape of the wound.

63. The wound care system as claimed in claim 59, in which the wound care system includes one or more supply apparatuses, for supplying gas and/or liquid to the wound bed, the one or more supply apparatuses connectable to the self-sealing valve connector via an opening connector, the one or more supply apparatuses being in the form of any of a fluid drip line, a gas line and a syringe, and in which the one or more supply apparatuses can be intermittently connected or disconnected from the self-sealing valve connector.

64. The wound care system as claimed in claim 59, in which the wound care system includes an extraction apparatus connectable to the self-sealing valve connector for extracting wound fluid and/or debris from the wound bed.

65. The wound care system as claimed in claim 60, in which the wound care system includes a reduced pressure source in the form of a vacuum pump connected to the reduced pressure port for providing negative pressure at the wound.

66. The wound care system as claimed in claim 61, in which the wound care system includes a reduced pressure source in the form of a vacuum pump connected to the reduced pressure port for providing negative pressure at the wound.
